(19) United States
(12) Patent Application Publication
(10) Pub. No.: US 2026/0201332 A1
(43) Pub. Date: Jul. 16, 2026

(54) METHOD FOR PLATELET DIFFERENTIATION, CULTURE MEDIUM, AND USE THEREOF

(71) Applicant: HEMACELL BIOTECHNOLOGY INC., Suzhou, Jiangsu (CN)

(72) Inventors: Chen WU, Suzhou, Jiangsu (CN); Chao LI, Suzhou, Jiangsu (CN)

(21) Appl. No.: 19/131,994

(22) PCT Filed: Nov. 24, 2022
(86) PCT No.: PCT/CN2022/133882

§ 371 Date: May 22, 2025

(51) Int. Cl. C12N 5/078

(52) U.S. Cl. CPC C12N 5/0644; C12N 2500/25; C12N 2500/32; C12N 2500/38; C12N 2501/125; C12N 2501/145; C12N 2501/155; C12N 2501/165; C12N 2501/2303; C12N 2501/2306; C12N 2501/26; C12N 2501/727; C12N 2501/734; C12N 2506/02; C12N 2506/11; C12N 2506/45

(57) ABSTRACT

The present application relates to a method for inducing proliferation and/or differentiation of pluripotent cells into platelets, which comprises using a culture medium comprising a specific added factor. The present application further provides a culture medium for inducing proliferation and/or differentiation of cells into platelets, and a use thereof.

### TECHNICAL FIELD

The present application relates to the field of biomedicine, and specifically to a method for differentiating platelets, a culture medium and a use thereof.

### BACKGROUND ART

Platelets are small circulating anucleated cells that are differentiated from mature megakaryocytes and contribute to hemostasis by aggregating and forming emboli in vascular injury. The life span of a single platelet in the body is about 7-10 days, so the human body produces a large number of fresh platelets every day to maintain a normal platelet count. As an inherent component of organisms, platelets may escape clearance by the immune system and are closely related to physiological processes such as vascular endothelial damage repair, immune response, atherosclerosis formation, neurodegeneration, tumor growth and metastasis, and have broad application prospects.

Platelet transfusion is a life-saving method used to prevent bleeding or stop ongoing bleeding in patients with thrombocytopenia and functional platelet disorders. It may also be used to prevent bleeding in patients with thrombocytopenia caused by chemotherapy for malignant tumors or hematopoietic stem cell transplantation. Currently, platelets mainly come from donations from donors. Due to the short storage time of donor-derived platelets and the insufficient number of donors, platelets have become a scarce resource. At the same time, there is a risk of bacterial contamination and deterioration in the process of platelet processing, preparation and transfusion. Therefore, it is very necessary to develop alternative strategies.

Pluripotent cells may proliferate and expand indefinitely in vitro and have the ability to differentiate and proliferate toward one or more blood cell lineages. Therefore, efficient platelet differentiation from pluripotent cells in vitro may be used as an effective method to alleviate the shortage of platelet supply, while reducing the risk of platelet contamination and deterioration.

### SUMMARY OF THE INVENTION

The present application provides a method for differentiating pluripotent cells and/or hematopoietic stem/progenitor cells and/or megakaryocytic progenitor cells into platelets. The method uses a platelet differentiation culture medium comprising N2 supplement and B27 supplement components, which may improve the differentiation ability of pluripotent cells, hematopoietic stem/progenitor cells, and megakaryocytic progenitor cells, and promote their differentiation into platelets.

In an aspect, the present application provides a method for inducing pluripotent cells to proliferate and/or differentiate through embryoids, hematopoietic stem/progenitor cells, megakaryocytic progenitor cells, and platelet pathways, the method comprising using a platelet differentiation culture medium comprising N2 supplement and B27 supplement components.

In an aspect, the present application provides a method for inducing hematopoietic stem/progenitor cells to proliferate and/or differentiate through megakaryocytic progenitor cells, and platelet pathways, the method comprising using a platelet differentiation culture medium comprising N2 supplement and B27 supplement components.

In some embodiments, the concentration of N2 supplement in the method is about 0.5%.

In some embodiments, the concentration of B27 supplement in the method is about 1%.

In some embodiments, the platelet differentiation culture medium in the method may further comprise one or more of TPO, SCF, IL6, ITS, glutamine, NEAA and ascorbic acid.

In some embodiments, the concentration of TPO in the method is about 5-100 ng/mL.

In some embodiments, the concentration of SCF in the method is about 5-100 ng/mL.

In some embodiments, the concentration of the IL6 in the method is about 5-100 ng/mL.

In some embodiments, the concentration of the ITS in the method is about 1%.

In some embodiments, the concentration of the glutamine in the method is about 1%.

In some embodiments, the concentration of the ascorbic acid in the method is about 50-200 μM.

In some embodiments, the platelet differentiation culture medium in the method may further comprise a basal culture medium, the culture medium may comprise serum, or the culture medium may be serum-free.

In some embodiments, the basal culture medium in the method may be one or more selected from the group consisting of IMDM, StemSpan™ SFEM II, APEL and mTeSR.

In some embodiments, the method may comprise adding one or more of the following substances to the platelet differentiation culture medium: a metalloproteinase inhibitor, a ROCK inhibitor.

In some embodiments, the metalloproteinase inhibitor in the method may be GM6001.

In some embodiments, the ROCK inhibitor in the method may be Y27632.

In some embodiments, the concentration of the Y27632 in the method is about 1-50 μM.

In some embodiments, the method may culture cells at about 37-39° C.

In some embodiments, the method may culture cells at about 5% CO₂.

In some embodiments, the method may culture cells in the absence of serum.

In some embodiments, the method may culture cells without co-culture.

In some embodiments, the method may culture cells on a surface coated with a matrix.

In some embodiments, the matrix in the method may be laminin, vitronectin, gelatin, polylysine, thrombospondin, or Matrigel™.

In some embodiments, the matrix in the method may be vitronectin.

In some embodiments, the matrix in the method may be Matrigel™.

In some embodiments, the method may comprise a step of culturing and/or maintaining the cells in a stem cell culture medium.

In some embodiments, the method may comprise a step of digesting cells that have proliferated to a sufficient number into single cells.

In some embodiments, the digestion step in the method may use Accutase digestive juice.

In some embodiments, the digestion step in the method may use Trypsin and EDTA.

In some embodiments, the pluripotent cells in the method may be derived from a mammal.

In some embodiments, the pluripotent cells in the method may be derived from a non-mammal.

In some embodiments, the pluripotent cells in the method may be human embryonic stem cells or human induced pluripotent stem cells.

In some embodiments, the hematopoietic stem/progenitor cells in the method may be derived from human embryonic stem cells, human induced pluripotent stem cells, ex vivo human blood, umbilical cord blood, and bone marrow.

In some embodiments, the hematopoietic stem/progenitor cells in the method may be CD34⁺ hematopoietic stem/progenitor cells.

In some embodiments, the megakaryocytic progenitor cells in the method may be derived from human embryonic stem cells, human induced pluripotent stem cells, ex vivo human blood, umbilical cord blood, bone marrow, and CD34⁺ hematopoietic stem/progenitor cells.

In another aspect, the present application provides a platelet differentiation culture medium, which comprises N2 supplement and B27 supplement components and may promote differentiation of cells into platelets.

In some embodiments, the concentration of N2 supplement in the culture medium is about 0.5%.

In some embodiments, the concentration of B27 supplement in the culture medium is about 1%.

In some embodiments, the culture medium may comprise platelet differentiation culture medium I. In some embodiments, the platelet differentiation culture medium I may comprise a basal culture medium, and the basal culture medium may be mTeSR.

In some embodiments, the platelet differentiation culture medium I may comprise an inhibitor. In some embodiments, the inhibitor may be a ROCK inhibitor. In some embodiments, the ROCK inhibitor may be Y27632, and the concentration of the Y27632 is about 1-50 μM.

In some embodiments, the culture medium may comprise platelet differentiation culture medium II. In some embodiments, the platelet differentiation culture medium II may comprise a basal culture medium, and the basal culture medium may be APEL.

In some embodiments, the platelet differentiation culture medium II may comprise one or more of BMP4, VEGF and SCF.

In some embodiments, the concentration of BMP4 in the platelet differentiation culture medium II is about 5-100 ng/mL.

In some embodiments, the concentration of VEGF in the platelet differentiation culture medium II is about 5-100 ng/mL.

In some embodiments, the concentration of SCF in the platelet differentiation culture medium II is about 5-100 ng/mL.

In some embodiments, the culture medium may comprise platelet differentiation culture medium III. In some embodiments, the platelet differentiation culture medium III may comprise a basal culture medium, and the basal culture medium may be StemSpan™ SFEM II.

In some embodiments, the platelet differentiation culture medium III may comprise one or more of IL3, IL6, SCF, TPO and FLt3L.

In some embodiments, the concentration of IL3 in the platelet differentiation culture medium III is about 5-100 ng/mL.

In some embodiments, the concentration of IL6 in the platelet differentiation culture medium III is about 5-100 ng/mL.

In some embodiments, the concentration of SCF in the platelet differentiation culture medium III is about 5-100 ng/mL.

In some embodiments, the concentration of TPO in the platelet differentiation culture medium III is about 5-100 ng/mL.

In some embodiments, the concentration of FLt3L in the platelet differentiation culture medium III is about 5-100 ng/mL.

In some embodiments, the culture medium may comprise platelet differentiation culture medium IV. In some embodiments, the platelet differentiation culture medium IV may comprise a basal culture medium, and the basal culture medium may be IMDM.

In some embodiments, the platelet differentiation culture medium IV may comprise one or more of N2 supplement, B27 supplement, NEAA, glutamine, ITS, ascorbic acid, IL6, SCF and TPO.

In some embodiments, the concentration of N2 supplement in the platelet differentiation culture medium IV is about 0.5%.

In some embodiments, the concentration of B27 supplement in the platelet differentiation culture medium IV is about 1%.

In some embodiments, the concentration of glutamine in the platelet differentiation culture medium IV is about 1%.

In some embodiments, the concentration of ITS in the platelet differentiation culture medium IV is about 1%.

In some embodiments, the concentration of ascorbic acid in the platelet differentiation culture medium IV is about 50-200 μM.

In some embodiments, the concentration of IL6 in the platelet differentiation culture medium IV is about 5-100 ng/mL.

In some embodiments, the concentration of SCF in the platelet differentiation culture medium IV is about 5-100 ng/mL.

In some embodiments, the concentration of TPO in the platelet differentiation culture medium IV is about 5-100 ng/mL.

In some embodiments, the culture medium comprises serum.

In some embodiments, the culture medium comprises serum.

In another aspect, the present application further provides use of the culture medium in inducing proliferation of pluripotent cells and/or differentiation of platelets.

In another aspect, the present application further provides use of the culture medium in inducing proliferation of hematopoietic stem/progenitor cells and/or differentiation of platelets.

In another aspect, the present application further provides use of the culture medium in inducing proliferation of megakaryocytic progenitor cells and/or differentiation of platelets.

In another aspect, the present application further provides a composition comprising pluripotent cells and the culture medium.

In another aspect, the present application further provides a composition comprising hematopoietic stem/progenitor cells and the culture medium.

In another aspect, the present application further provides a composition comprising megakaryocytic progenitor cells and the culture medium.

In another aspect, the present application further provides a culture platform for obtaining platelets, which comprises the method and the culture medium.

In another aspect, the present application further provides use of the culture platform. In some embodiments, the use comprises screening other compounds.

In another aspect, the present application further provides use of the method, the culture medium, the composition and/or the culture platform in preparation of platelets.

Those skilled in the art may easily discern other aspects and advantages of the present application from the following detailed description. Only exemplary embodiments of the present application are shown and described in the following detailed description. As those skilled in the art will realize, the contents of the present application enable those skilled in the art to make changes to the specific embodiments disclosed without departing from the spirit and scope of the invention covered by the present application. Accordingly, the drawings and descriptions in the specification of the present application are illustrative only and not restrictive.

### BRIEF DESCRIPTION OF DRAWINGS

The specific features of the invention to which the present application relates are set forth in the appended claims. The features and advantages of the invention to which the present application relates may be better understood by reference to the exemplary embodiments described in detail below and the drawings. A brief description of the drawings is as follows:

FIG. 1 shows the flow cytometry analysis results when the platelet differentiation culture medium IV in the present application is S1.

FIG. 2 shows the flow cytometry analysis results when the platelet differentiation culture medium IV in the present application is S2.

FIG. 3 shows the flow cytometry analysis results when the platelet differentiation culture medium IV in the present application is S3.

FIG. 4 shows the flow cytometry analysis results when the platelet differentiation culture medium IV in the present application is S4.

FIG. 5 shows the flow cytometry analysis results when the platelet differentiation culture medium IV in the present application is S5.

FIG. 6 shows the flow cytometry analysis results when the platelet differentiation culture medium IV in the present application is S6.

### DETAILED DESCRIPTION

The embodiments of the invention of the present application will be described below with specific examples. Those skilled in the art may easily understand other advantages and effects of the invention of the present application from the disclosure of the specification.

### DEFINITION OF TERMS

In the present application, the term “pluripotent cell” generally refers to a cell having the potential for proliferation and differentiation. Pluripotent cells may be functionally defined as cells that: (1) have the ability to differentiate into different cell types and, in some cases, generate only one specialized cell type, and (2) are capable of long-term self-renewal, generating one or more cells that may be the same or different from the original cell type. The source and preparation method of pluripotent cells are not limited. For example, the pluripotent cells may be naturally obtained or artificially modified. For example, the pluripotent cells may include embryonic stem cells, induced pluripotent stem cells, and the like.

In the present application, the term “proliferation” generally refers to the generation of multiple individual cells by the division of an initial cell. The multiple individual cells may be cells of the same type or cells of different types. The initial cells used for proliferation need not be the same as the cells resulting from the proliferation. For example, the proliferated cells may result from the growth and differentiation of a starting population of cells.

In the present application, the term “differentiation” generally refers to the process by which non-specific or less specific cells acquire the characteristic of particular cells. Differentiated or differentiation-induced cells are cells that have taken up a more specific position in a cell lineage.

In the present application, the term “megakaryocytic progenitor cell” is also called “megakaryocyte”, which generally refers to a cell that may produce platelets. Megakaryocytic progenitor cells feature large polyploid nuclei, large cell size, and rich cytoplasm, allowing each cell to produce thousands of platelets. The source and preparation method of megakaryocytic progenitor cells are not limited. For example, the megakaryocytic progenitor cells may be differentiated from pluripotent cells or isolated in vivo.

In the present application, the term “embryonic stem cell”, abbreviated as “ESC”, generally refers to cells characterized by unlimited proliferation, self-renewal and multidirectional differentiation. Embryonic stem cells are stem cells obtained from the undifferentiated inner cell mass of the blastocyst (early embryonic stage). There is no restriction on their source and preparation method. Embryonic stem cells may be induced to differentiate into almost all cell types of the organism, whether in vitro or in vivo. For example, the cell types may be hematopoietic stem cells, nerve cells, cardiomyocytes, etc.

In the present application, the term “induced pluripotent stem cell” may be abbreviated as iPS cell or iPSC, and generally refers to a class of pluripotent stem cells prepared by an artificial means from non-pluripotent cells. Induced pluripotent stem cells may be obtained by introducing specific transcription factors to reprogram terminally differentiated somatic cells. For example, the terminally differentiated somatic cells may be fibroblasts, hematopoietic stem cells, muscle cells, neurons, epidermal cells and the like.

In the present application, the term “hematopoietic stem cells” generally refers to cells that have the ability of long-term self-renewal and the potential to differentiate into various types of mature blood cells. The source and preparation method of hematopoietic stem cells are not limited. For example, the hematopoietic stem cells may be differentiated from pluripotent cells and may be separated from bone marrow or blood. Hematopoietic stem cells may differentiate into many types of cells, for example, bone marrow lineage cells (e.g. monocytes and macrophages, neutrophils, basophils, eosinophils, red blood cells, megakaryocytes/platelets, dendritic cells), and lymphoid lineage cells (e.g. T-cells, B-cells, NK-cells).

In the present application, the term “platelet” generally refers to an acaryotic cytosome. Platelets may be formed by small pieces of cytoplasm that fall off during the lysis of megakaryocytic progenitor cells, and they play an important role in the physiological hemostasis process. They may be activated by thrombin, quickly adhere to the wound, and aggregate into clumps to form softer hemostatic plugs, then promote blood coagulation and form solid hemostatic plugs.

In the present application, the term “composition” generally refers to products comprising specified amounts of specified ingredients, and any products produced directly or indirectly from a combination of specified amounts of specified ingredients. In the present application, the composition may further comprise other inactive ingredients, for example, carriers, excipients, adjuvants, and stabilizers and the like.

In the present application, the term “ex vivo” generally refers to the manipulation of cells, tissues and/or organs that have been removed from the body of the organism. In some embodiments, the cells, tissues and/or organs may be returned to the organism by certain methods, or introduced into another organism.

In the present application, the term “in vitro” generally refers to the removal or release of a portion of an organism from the organism.

In the present application, the term “comprising” generally means to including, generalizing, containing or covering. In some cases, it also means “being” and “consisting of”.

In the present application, the terms “and/or” shall be understood to mean any one, two, or more of the alternatives, or any combination thereof.

In the present application, the term “about” typically refers to a change in a range of 0.5-10% greater or less than a specified value, e.g., a change in a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% greater or less than the specified value.

### DETAILED DESCRIPTION OF THE INVENTION

### Methods

In an aspect, the present invention provides a method for inducing proliferation and/or differentiation of pluripotent cells into platelets, comprising using a platelet differentiation culture medium comprising N2 supplement and B27 supplement components.

In another aspect, the present invention provides a method for inducing proliferation and/or differentiation of hematopoietic stem/progenitor cells into platelets, comprising using a platelet differentiation culture medium comprising N2 supplement and B27 supplement components.

In another aspect, the present invention provides a method for inducing proliferation and/or differentiation of megakaryocytic progenitor cells into platelets, comprising using a platelet differentiation culture medium comprising N2 supplement and B27 supplement components.

In the present application, the method may further comprise the process of culturing, proliferating and/or differentiating pluripotent cells; the method may further comprise the process of culturing, proliferating and/or differentiating embryoids; the method may further comprise the process of culturing, proliferating and/or differentiating hematopoietic stem/progenitor cells; the method may further comprise the process of culturing, proliferating and/or differentiating megakaryocytic progenitor cells; the method may further comprise the process of culturing platelets; the method may further comprise the process of proliferating and/or differentiating pluripotent cells into embryoids; the method may further comprise the process of proliferating and/or differentiating pluripotent cells into hematopoietic stem/progenitor cells; the method may further comprise the process of proliferating and/or differentiating pluripotent cells into megakaryocytic progenitor cells; the method may further comprise the process of differentiating embryoids into hematopoietic stem/progenitor cells; the method may further comprise the process of differentiating embryoids into megakaryocytic progenitor cells; the method may further comprise the process of differentiating embryoids into platelets; the method may further comprise the process of proliferating and/or differentiating hematopoietic stem/progenitor cells into megakaryocytic progenitor cells.

In the present application, the culture method may comprise the following steps: (1) seeding pluripotent cells into platelet differentiation culture medium I to obtain embryoids; (2) seeding embryoids into platelet differentiation culture medium II to obtain hematopoietic stem/progenitor cells; (3) seeding hematopoietic stem/progenitor cells into platelet differentiation culture medium III to obtain megakaryocytic progenitor cells; (4) seeding megakaryocytic progenitor cells into platelet differentiation culture medium IV to obtain platelets.

In the present application, the culture method may comprise the following steps: (1) seeding pluripotent cells into platelet differentiation culture medium I and culturing the cells for about 5-8 days to obtain embryoids; (2) seeding embryoids into platelet differentiation culture medium II and culturing the cells for about 7 days to obtain hematopoietic stem/progenitor cells; (3) seeding hematopoietic stem/progenitor cells into platelet differentiation culture medium III and culturing the cells for about 7 days to obtain megakaryocytic progenitor cells; (4) seeding megakaryocytic progenitor cells into platelet differentiation culture medium IV and culturing the cells for about 5-10 days to obtain platelets.

In the present application, the culture method may comprise the following steps: (1) seeding hematopoietic stem/progenitor cells in platelet differentiation culture medium III to obtain megakaryocytic progenitor cells; (2) seeding megakaryocytic progenitor cells in platelet differentiation culture medium IV to obtain platelets.

In the present application, the culture method may comprise the following steps: (1) seeding hematopoietic stem/progenitor cells in platelet differentiation culture medium III and culturing the cells for about 7 days to obtain megakaryocytic progenitor cells; (2) seeding megakaryocytic progenitor cells in platelet differentiation culture medium IV and culturing the cells for about 5-10 days to obtain platelets.

In the present application, the culture method may comprise culturing cells on a surface coated with a matrix.

In some embodiments, the matrix in the culture method may be laminin, vitronectin, gelatin, polylysine, thrombospondin, or Matrigel™. In some embodiments, the matrix in the culture method may be vitronectin or Matrigel™ In the present application, the culture method may comprise regularly or irregularly supplementing and/or replacing the culture medium.

In the present application, the culture method may comprise digesting the cells into single cells after the cells proliferate to a sufficient number.

In some embodiments, the digestion in the culture method uses Accutase digestive juice. In some embodiments, the digestion in the culture method uses Trypsin and EDTA.

In the present application, the culture method may comprise culturing cells at about 37-39° C., for example, about 36.5° C., about 37° C., about 37.5° C., about 38° C., about 38.5° C., about 39° C., about 39.5° C.

In the present application, the culture method may comprise culturing cells at about 3-7% CO₂, for example, about 3% CO₂, about 3.5% CO₂, about 4% CO₂, about 4.5% CO₂, about 5% CO₂, about 5.5% CO₂, about 6% CO₂, about 6.5% CO₂, about 7% CO, about 7.5% CO₂.

In the present application, the culture method may be carried out under culture conditions with serum.

In the present application, the culture method may be carried out under culture conditions without serum.

In the present application, the culture method may be carried out under culture conditions without feeding.

In the present application, the culture method may be carried out under culture conditions with feeding.

In the present application, the source of the pluripotent cells in the culture method is not limited, and may be of mammalian origin or non-mammalian origin.

In some embodiments, the pluripotent cells are derived from human embryonic stem cells. In some embodiments, the pluripotent cells are derived from human induced pluripotent stem cells.

In the present application, the source of hematopoietic stem/progenitor cells in the culture method is not limited.

In some embodiments, the hematopoietic stem/progenitor cells are derived from human embryonic stem cells. In some embodiments, the hematopoietic stem/progenitor cells are derived from human induced pluripotent stem cells. In some embodiments, the hematopoietic stem/progenitor cells are derived from ex vivo human blood. In some embodiments, the hematopoietic stem/progenitor cells are derived from umbilical cord blood. In some embodiments, the hematopoietic stem/progenitor cells are derived from bone marrow. In some embodiments, the hematopoietic stem/progenitor cells are CD34⁺ hematopoietic stem/progenitor cells.

In the present application, the source of megakaryocytic progenitor cells in the culture method is not limited.

In some embodiments, the megakaryocytic progenitor cells are derived from human embryonic stem cells. In some embodiments, the megakaryocytic progenitor cells are derived from human induced pluripotent stem cells. In some embodiments, the megakaryocytic progenitor cells are derived from ex vivo human blood. In some embodiments, the megakaryocytic progenitor cells are derived from umbilical cord blood. In some embodiments, the megakaryocytic progenitor cells are derived from bone marrow. In some embodiments, the megakaryocytic progenitor cells are derived from CD34⁺ hematopoietic stem/progenitor cells.

In the present application, one or more substances may be added into the culture medium in the culture method, including but not limited to: nutrients/extracts, growth factors, hormones, cytokines and culture medium supplements.

In some embodiments, the culture medium in the culture method may comprise a basal culture medium for culturing cells. The basal culture medium may be a single component or a combination of multiple culture media. The basal culture medium includes but is not limited to IMDM, MEM, Ham's F12, mTeSR1, APEL, StemSpan™ SFEM II, DMEM, and RPMI1640.

In some embodiments, one or more of the following substances may be added into the culture medium in the culture method, including but not limited to serum replacement, glutamine, NEAA (non-essential amino acids), ascorbic acid, epidermal growth factor (EGF), acidic fibroblast growth factor (aFGF), basic fibroblast growth factor (bFGF), leukemia inhibitory factor (LIF), hepatocyte growth factor (HGF), insulin-like growth factor 1 (IGF-1), insulin-like growth factor 2 (IGF-2), keratinocyte growth factor (KGF), nerve growth factor (NGF), platelet-derived growth factor (PDGF), transforming growth factor β (TGF-β), bone morphogenetic protein (BMP4), vascular endothelial growth factor (VEGF), transferrin, insulin, selenium, various interleukins (e.g. IL-1 to IL-18), various colony stimulating factors (e.g. granulocyte/macrophage colony stimulating factor (GM-CSF)), various interferons (e.g. IFN-γ), stem cell factor (SCF), thrombopoietin (TPO), erythropoietin (EPO), N2 supplement, B27 supplement, Fms-related tyrosine kinase 3 ligand (FLt3L). The added substances may be from any source and may be commercially available, natural or recombinant.

In some embodiments, inhibitors may be added to the culture medium in the culture method, and the inhibitors may include but are not limited to GSK-3 inhibitors, MEK inhibitors, ROCK inhibitors, metalloproteinase inhibitors, and the like.

In some embodiments, the ROCK inhibitor includes but is not limited to polynucleotides, polypeptides and small molecules, which may reduce ROCK expression and/or ROCK activity. In some embodiments, the ROCK inhibitor may be Y27632. In some embodiments, the metalloproteinase inhibitor may be GM6001.

### Culture Medium

In another aspect, the present application provides a platelet differentiation culture medium that may be used for proliferation and/or differentiation of cells and may be used for differentiation of platelets.

In some embodiments, the culture medium may be a single component, comprising N2 supplement and B27 supplement.

In some embodiments, the culture medium may be a combination culture medium, one or more of which may be used to perform the relevant operations, and one or more of the culture media in the combination culture medium may comprise N2 supplement and B27 supplement.

In some embodiments, one or more substances may be added into the culture medium, including but not limited to: nutrients/extracts, growth factors, hormones, cytokines and culture medium supplements.

In some embodiments, the culture medium may comprise a basal culture medium for culturing cells. The basal culture medium may be a single component or a combination of multiple culture media. The basal culture medium includes but is not limited to IMDM, MEM, Ham's F12, mTeSR1, APEL, StemSpan™ SFEM II, DMEM, and RPMI1640.

In some embodiments, one or more of the following substances may be added into the culture medium, including but not limited to serum replacement, glutamine, NEAA (non-essential amino acids), ascorbic acid, epidermal growth factor (EGF), acidic fibroblast growth factor (aFGF), basic fibroblast growth factor (bFGF), leukemia inhibitory factor (LIF), hepatocyte growth factor (HGF), insulin-like growth factor 1 (IGF-1), insulin-like growth factor 2 (IGF-2), keratinocyte growth factor (KGF), nerve growth factor (NGF), platelet-derived growth factor (PDGF), transforming growth factor β (TGF-β), bone morphogenetic protein (BMP4), vascular endothelial growth factor (VEGF), transferrin, insulin, selenium, various interleukins (e.g. IL-1 to IL-18), various colony stimulating factors (e.g. granulocyte/macrophage colony stimulating factor (GM-CSF)), various interferons (e.g. IFN-γ), stem cell factor (SCF), thrombopoietin (TPO), erythropoietin (EPO), N2 supplement, B27 supplement, Fms-related tyrosine kinase 3 ligand (FLt3L). The added substances may be from any source and may be commercially available, natural or recombinant.

In some embodiments, inhibitors may be added to the culture medium, and the inhibitors may include but are not limited to GSK-3 inhibitors, MEK inhibitors, ROCK inhibitors, metalloproteinase inhibitors, and the like.

In some embodiments, the ROCK inhibitor includes but is not limited to polynucleotides, polypeptides and small molecules, which may reduce ROCK expression and/or ROCK activity. In some embodiments, the ROCK inhibitor can be Y27632, Thiazovivin, Fasudil (HA-1077), GSK429286A, RKI-1447, WAY-624704, H-1152, Azaindole 1 (TC-S 7001), Hydroxyfasudil (HA-1100), Y-39983, Netarsudil (AR-13324), GSK269962A, Ripasudil (K-115) hydrochloride dihydrate, Belumosudil (KD025), AT13148, Emetine hydrochloride, ZINC00881524 or a ROCK pathway-targeted antibody.

In some embodiments, the metalloproteinase inhibitor may be GM6001, CL-82198, SM-7368, Dendrobii caulis Extract, T-26c, AUDA (compound 43), MMP-9-IN-1 (OUN87710), Isoliquiritin apioside (ISLA, ILA), Nobiletin (NSC 76751, Hexamethoxyflavone), Solasodine, Triolein, Cordycepin (3′-Deoxyadenosine), Doxycycline Hyclate (WC2031), Trans-Zeatina ((E)-Zeatin), Lactobionic acid (Galactosylgluconic acid), Doxycycline, Morroniside, 1,10-Phenanthroline, JNJ0966, Abametapir (HA-44, BRN 0123183), o-Phenanthroline, Batimastat (BB-94), Marimastat (BB-2516, TA2516), SB-3CT, TAPI-1, NSC 405020, T-5224, Auraptene (7-geranyloxycoumarin), Polygalacic acid, ARP 100, Absinthin (Absynthine), Ginkgolide C (BN-52022).

In the present application, the culture medium in the method and the platelet differentiation culture medium may be a combination of one or more of the following culture media: platelet differentiation culture medium I, platelet differentiation culture medium II, platelet differentiation culture medium III, platelet differentiation culture medium IV.

In some embodiments, the platelet differentiation culture medium I may comprise a basal culture medium, for example, mTeSR.

In some embodiments, the platelet differentiation culture medium I may comprise a basal culture medium and other substances, for example, a ROCK inhibitor. In some embodiments, the ROCK inhibitor can be Y27632.

In some embodiments, the platelet differentiation culture medium II may comprise a basal culture medium, for example, APEL.

In some embodiments, the platelet differentiation culture medium II may comprise a basal culture medium and other substances. For example, the other substances may include one or more selected from the group consisting of BMP4, VEGF, SCF.

In some embodiments, the platelet differentiation culture medium III may comprise a basal culture medium, for example, StemSpan™ SFEM II.

In some embodiments, the platelet differentiation culture medium III may comprise a basal culture medium and other substances. For example, the other substances may include one or more selected from the group consisting of IL3, IL6, SCF, TPO, FLt3L.

In some embodiments, the platelet differentiation culture medium IV may comprise a basal culture medium, for example, IMDM.

In some embodiments, the platelet differentiation culture medium IV may include a basal culture medium and other substances. For example, the other substances may include one or more selected from the group consisting of N2 supplement, B27 supplement, NEAA, glutamine, ITS, ascorbic acid, IL6, SCF, TPO.

In the present application, in the culture medium in the method and the platelet differentiation culture medium, the concentration of the N2 supplement may be about 0.5%. For example, the concentration of the N2 supplement is about 0.25%, about 0.26%, about 0.27%, about 0.28%, about 0.29%, about 0.3%, about 0.31%, about 0.32%, about 0.33%, about 0.34%, about 0.35%, about 0.36%, about 0.37%, about 0.38%, about 0.39%, about 0.4%, about 0.41%, about 0.42%, about 0.43%, about 0.44%, about 0.45%, about 0.45%, about 0.47%, about 0.48%, about 0.49%, about 0.5%, about 0.51%, about 0.52%, about 0.53%, about 0.54%, about 0.55%, about 0.56%, about 0.57%, about 0.58%, about 0.59%, about 0.6%, about 0.7%, about 0.71%, about 0.72%, about 0.73%, about 0.74%, about 0.75%, about 0.76%, about 0.77%, about 0.78%, about 0.79%, about 0.8%.

In the present application, in the culture medium in the method and the platelet differentiation culture medium, the concentration of the B27 supplement may be about 1%. For example, the concentration of the B27 supplement is about 0.25%, about 0.3%, about 0.35%, about 0.40%, about 0.45%, about 0.5%, about 0.55%, about 0.6%, about 0.65%, about 0.7%, about 0.75%, about 0.8%, about 0.85%, about 0.9%, about 0.95%, about 1%, about 1.05%, about 1.1%, about 1.15%, about 1.2%, about 1.25%, about 1.3%, about 1.35%, about 1.4%, about 1.45%, about 1.5%, about 1.55%, about 1.6%, about 1.65%, about 1.7%, about 1.75%.

In the present application, in the culture medium in the method and the platelet differentiation culture medium, the concentration of the Y27632 may be about 1-50 μM. For example, the Y27632 concentration is about 1 μM, about 3 μM, about 5 μM, about 8 μM, about 10 μM, about 15 μM, about 20 μM, about 25 μM, about 30 μM, about 35 μM, about 40 μM, about 45 μM, about 50 μM.

In the present application, in the culture medium in the method and the platelet differentiation culture medium, the concentration of the BMP4 may be about 5-100 ng/mL. For example, the concentration of the BMP4 is about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL.

In the present application, in the culture medium in the method and the platelet differentiation culture medium, the concentration of the VEGF may be about 5-100 ng/mL. For example, the concentration of the VEGF is about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL.

In the present application, in the culture medium in the method and the platelet differentiation culture medium, the concentration of the SCF may be about 5-100 ng/mL. For example, the concentration of the SCF is about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL.

In the present application, in the culture medium in the method and the platelet differentiation culture medium, the concentration of the IL3 may be about 5-100 ng/mL. For example, the concentration of the IL3 is about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL.

In the present application, in the culture medium in the method and the platelet differentiation culture medium, the concentration of the IL6 may be about 5-100 ng/mL. For example, the concentration of the IL6 is about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL.

In the present application, in the culture medium in the method and the platelet differentiation culture medium, the concentration of the TPO may be about 5-100 ng/mL. For example, the concentration of the TPO is about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL.

In the present application, in the culture medium in the method and the platelet differentiation culture medium, the concentration of the FLt3L may be about 5-100 ng/mL. For example, the 15 concentration of the FLt3L is about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL.

In the present application, in the culture medium in the method and the platelet differentiation culture medium, the concentration of the glutamine may be about 1%. For example, the concentration of the glutamine is about 0.25%, about 0.3%, about 0.35%, about 0.40%, about 0.45%, about 0.5%, about 0.55%, about 0.6%, about 0.65%, about 0.7%, about 0.75%, about 0.8%, about 0.85%, about 0.9%, about 0.95%, about 1%, about 1.05%, about 1.1%, about 1.15%, about 1.2%, about 1.25%, about 1.3%, about 1.35%, about 1.4%, about 1.45%, about 1.5%, about 1.55%, about 1.6%, about 1.65%, about 1.7%, about 1.75%.

In the present application, in the culture medium in the method and the platelet differentiation culture medium, the concentration of the ITS may be about 1%. For example, the concentration of the ITS is about 0.25%, about 0.3%, about 0.35%, about 0.40%, about 0.45%, about 0.5%, about 0.55%, about 0.6%, about 0.65%, about 0.7%, about 0.75%, about 0.8%, about 0.85%, about 0.9%, about 0.95%, about 1%, about 1.05%, about 1.1%, about 1.15%, about 1.2%, about 1.25%, about 1.3%, about 1.35%, about 1.4%, about 1.45%, about 1.5%, about 1.55%, about 1.6%, about 1.65%, about 1.7%, about 1.75%.

In the present application, in the culture medium in the method and the platelet differentiation culture medium, the concentration of the ascorbic acid may be about 50-200 μM. For example, the concentration of the ascorbic acid is about 50 μM, about 55 μM, about 60 μM, about 65 μM, about 70 μM, about 75 μM, about 80 μM, about 85 μM, about 90 μM, about 95 μM, about 100 μM, about 105 μM, about 110 μM, about 115 μM, about 120 μM, about 125 μM, about 130 μM, about 135 μM, about 140 μM, about 145 μM, about 150 μM, about 155 μM, about 160 μM, about 165 μM, about 170 μM, about 175 μM, about 180 μM, about 185 μM, about 190 μM, about 195 μM, about 200 μM.

### Composition and Use

In another aspect, the present application provides a composition comprising pluripotent cells and the culture medium.

In another aspect, the present application provides a composition comprising hematopoietic stem/progenitor cells and the culture medium.

In another aspect, the present application provides a composition comprising megakaryocytic progenitor cells and the culture medium.

In some embodiments, the ingredients and contents of the composition may vary. In some embodiments, the ingredients and contents of the composition may be specified. In some embodiments, the composition may comprise other inactive ingredients, for example, carriers, excipients, adjuvants, and stabilizers.

In another aspect, the present application provides use of the method in differentiation of platelets, which comprises the culture medium.

In another aspect, the present application provides use of the culture medium in inducing proliferation of pluripotent cells and/or differentiation of platelets.

In another aspect, the present application provides use of the culture medium in proliferation of hematopoietic stem/progenitor cells and/or differentiation of platelets.

In another aspect, the present application provides use of the culture medium in inducing proliferation of megakaryocytic progenitor cells and/or differentiation of platelets.

In another aspect, the present application provides a culture platform for obtaining platelets, which comprises the method and the culture medium.

In another aspect, the present application provides use of the culture platform, which comprises the method and the culture medium.

In another aspect, the present application provides use of the method, the culture medium, the composition and the culture platform in preparation of platelets.

In another aspect, the present application provides a method for preventing and/or treating a disease, comprising using the method, the culture medium, and the composition.

Without wishing to be bound by any theory, the following examples are only for illustrating the various technical solutions of the present application, and are not used to limit the scope of the invention of the present application.

### EXAMPLES

### Example 1 Differentiation and Culture of Platelets

Cells were induced to differentiate into platelets using the following methods and culture media:
(1) ESC/iPSC was seeded in Matrigel or vitronectin pre-coated culture dishes or six-well cell culture plates (1×10⁶ cells per 10 cm culture dish or 2×10⁵ cells per well of the 6-well plate), and cultured in mTeSR1 culture medium at 37° C., 5% CO₂ until the cell saturation became about 70-80%, which usually took about 4-7 days;
(2) The culture supernatant was removed, and the cells were washed once with DPBS buffer preheated to 37° C.; digested with Accutase digestive juice at 37° C. for about 5 min, and resuspended and collected in preheated DPBS;
(3) On Day 0, the cell suspension was centrifuged at 300 g for 5 min, the supernatant was discarded, and the cells were resuspended in platelet differentiation culture medium I and seeded into a 6-well suspension culture plate (3×10⁵ cells per well of the 6-well plate) and cultured at 37° C., 5% CO₂; (4) On Day 1, the cell suspension was collected and centrifuged at 300 g for 5 min, the supernatant was removed, the cells were then cultured with platelet differentiation culture medium II at 37° C., 5% CO₂;
(5) From Day 1 to Day 6, the cells were cultured continuously and fresh culture medium was replaced regularly;
(6) On Day 7, the EB suspension was collected and centrifuged at 400 g for 5 min, the supernatant was removed, the cells were digested with 1 ml of preheated 0.05% Trypsin+EDTA in a 37° C. water bath for 5 min, then 1 ml of 10% FBS was added to terminate the digestion and the cells were centrifuged at 400 g for 5 min;
(7) The supernatant of the cell suspension was removed and the cells were resuspended in differentiation culture medium III to obtain CD34⁺ hematopoietic stem/progenitor cells; this step may give CD34⁺ cells with a purity greater than 90% as analyzed by flow cytometry;
(8) From Day 7 to Day 14, the CD34⁺ hematopoietic stem/progenitor cell suspension was seeded into a 6-well suspension culture plate (8×10⁴ cells per well) and cultured at 37° C., 5% CO₂ for 7 days, half of the medium was replaced regularly with fresh platelet differentiation culture medium III; (9) On Day 14, megakaryocytic progenitor cells (MKPs) may be obtained, MKPs were collected, centrifuged at 400 g for 10 min, the supernatant was discarded, and the cells were resuspended in platelet differentiation culture medium IV, seeded into a 6-well suspension culture plate (8×10⁵ cells per well), and cultured at 37° C., 5% CO₂;
(10) From Day 14 to Day 19, the cells were continuously cultured and regularly supplemented with fresh platelet differentiation culture medium IV;
(11) On Day 19, fresh platelet differentiation culture medium IV was supplemented, and the cells were transferred to culture at 39° C., 5% CO₂, and fresh platelet differentiation culture medium IV was supplemented regularly;
(12) On Day 22, GM6001 was added to each well and culture was continued at 39° C., 5% CO₂ for 2 days;
(13) On Day 24, after the cells were collected, they were centrifuged at 1000 g for 10 min to obtain a high-purity platelet suspension;
(14) During the culture of MKPs, the culture medium components were optimized and improved, and platelet differentiation culture medium IV with different components was used, the components of which are shown in Table 1;
(15) Platelet differentiation culture medium I: may comprise mTeSR basal culture medium, ROCK inhibitor Y27632 at a concentration of about 1-50 μM;
(16) Platelet differentiation culture medium II: may comprise APEL basal culture medium, with BMP4 at a concentration of about 5-100 ng/mL, VEGF at a concentration of about 5-100 ng/mL, and SCF concentration of about 5-100 ng/mL;
(17) Platelet differentiation culture medium III: may comprise StemSpan™ SFEM II medium, with IL3 at a concentration of about 5-100 ng/mL, IL6 at a concentration of about 5-100 ng/mL, SCF at a concentration of about 5-100 ng/mL, TPO at a concentration of about 5-100 ng/mL, and FLt at a concentration of about 5-100 ng/mL;
(18) Platelet differentiation culture medium IV: may comprise IMDM basal culture medium, with N2 supplement at a concentration of about 0.5%, B27 supplement at a concentration of about 1%, glutamine at a concentration of about 1%, ITS at a concentration of about 1%, ascorbic acid at a concentration of about 50-200 μM, IL6 at a concentration of about 5-100 ng/mL, SCF at a concentration of about 5-100 ng/mL, TPO at concentration of about 5-100 ng/mL, as well as NEAA.

TABLE 1
Different differentiation systems of platelet differentiation
culture medium IV

| | | | | | | | IL6 + |
|---|---|---|---|---|---|---|---|
| | | | N2 + | ITS | NEAA + | Ascorbic | SCF + |
| Group | IMDM | BSA | B27 | | glutamine | acid | TPO |
| S1 | √ | √ | √ | √ | √ | √ | √ |
| S2 | √ | | √ | √ | √ | √ | √ |
| S3 | √ | √ | | √ | √ | √ | √ |
| S4 | √ | √ | √ | | √ | √ | √ |
| S5 | √ | √ | √ | √ | | √ | √ |
| S6 | √ | √ | √ | √ | √ | | √ |

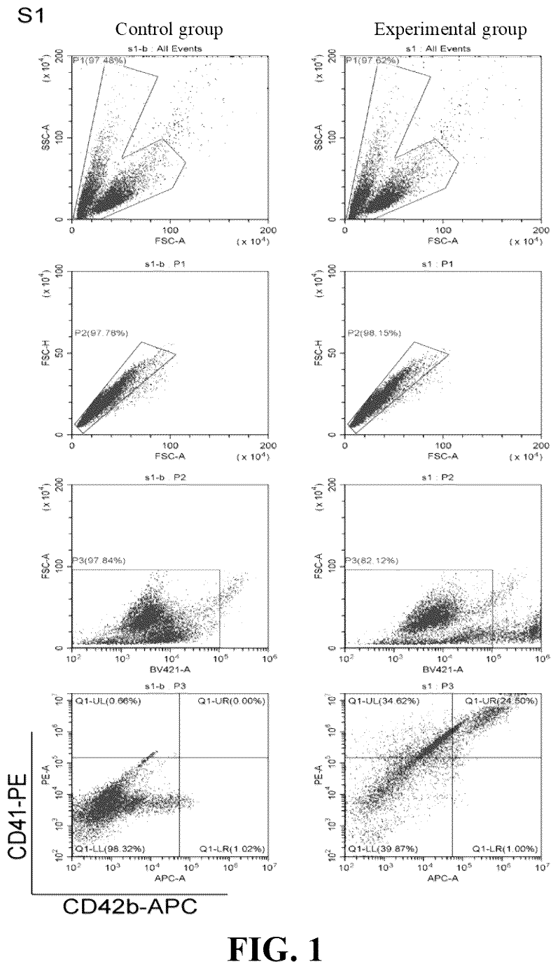

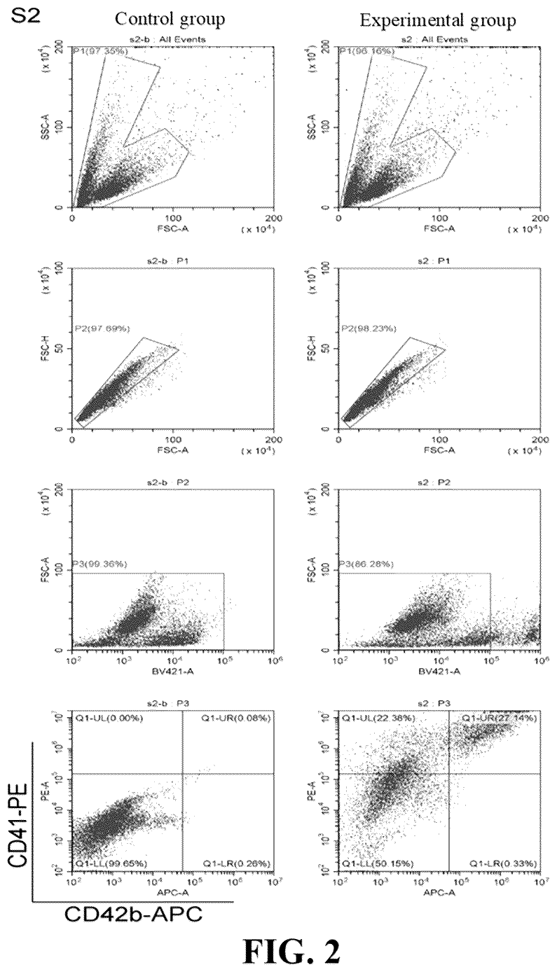

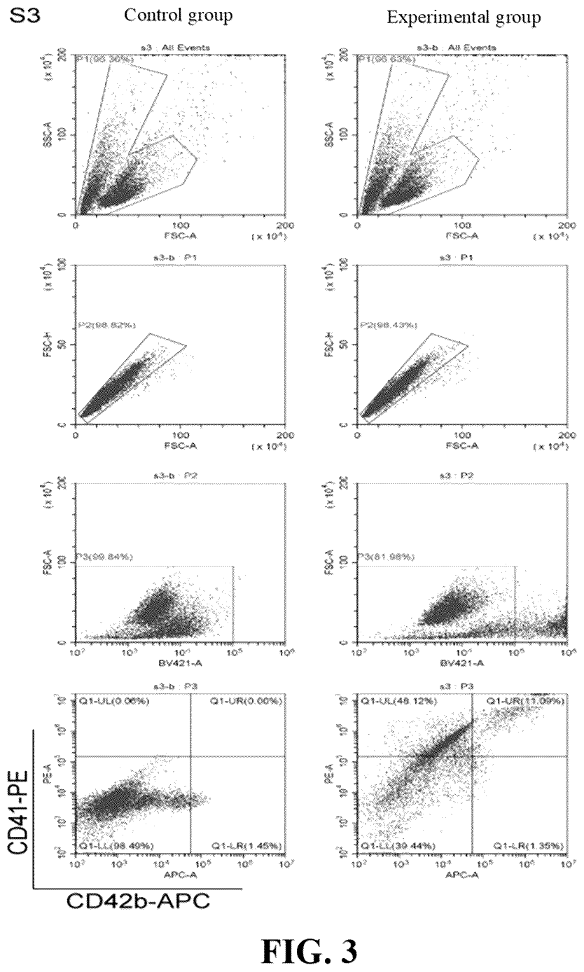

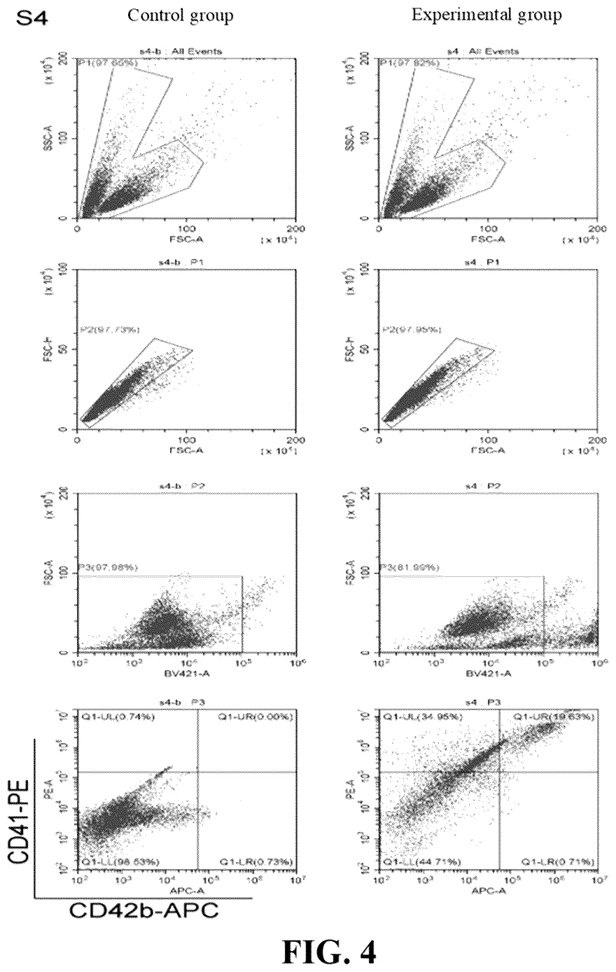

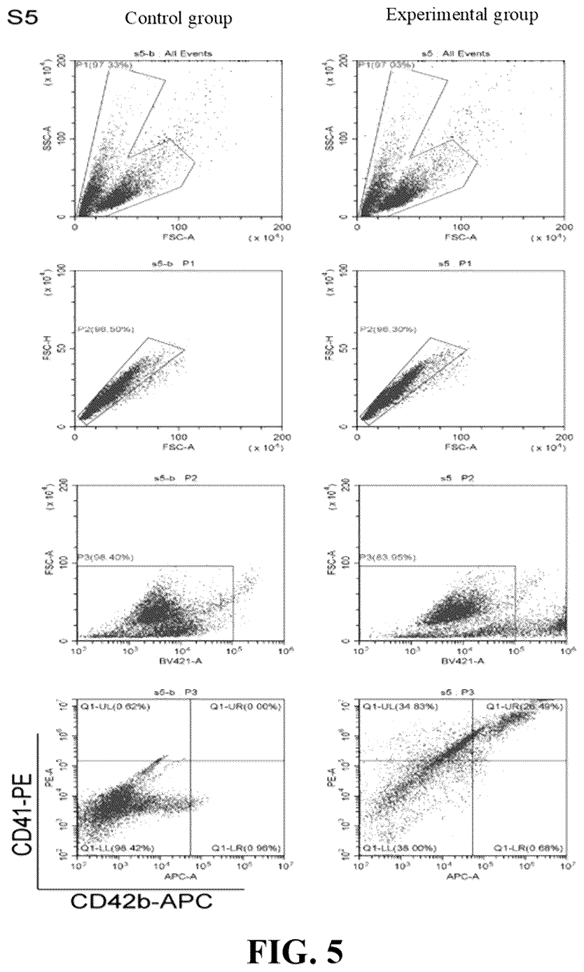

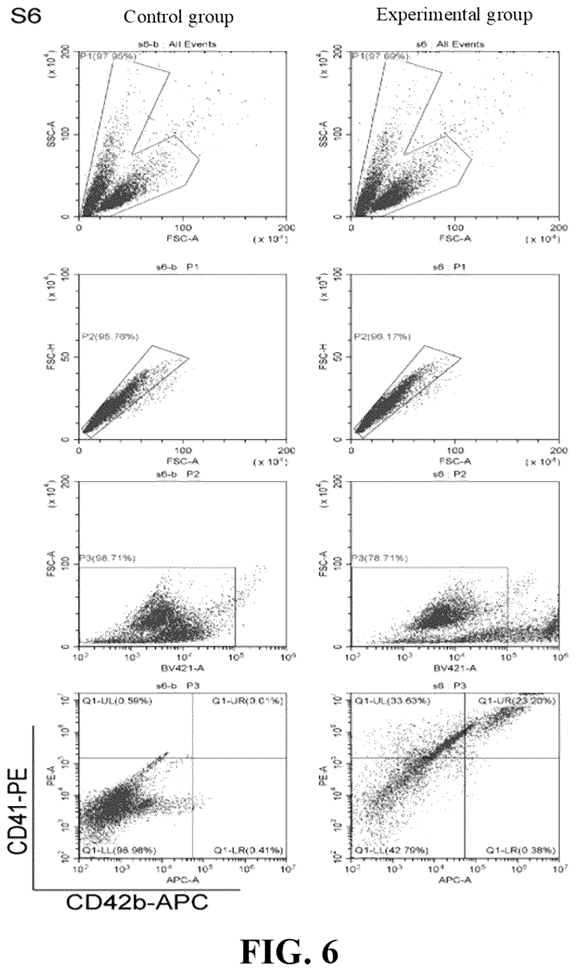

The experimental results are shown in FIGS. 1 to 6. Cell surface protein staining (\textcircled{1} PE-CD41; APC-CD42b; DAPI \textcircled{2} APC-CD61; FITC-CD63; DAPI) was performed, and then flow cytometry analysis was performed. The statistical results are shown in Table 2, indicating that N2 supplement and B27 supplement have a decisive effect on platelet differentiation.

TABLE 2
Flow cytometry result statistics

| | | | | | | CD61⁺ |
|---|---|---|---|---|---|---|
| Group | CD41⁺ | CD42b⁺ | CD41⁺CD42b⁺ | CD61⁺ | CD63⁺ | CD63⁺ |
| S1 | 59.12% | 25.50% | 24.50% | 60.65% | 33.72% | 26.95% |
| S2 | 49.52% | 27.47% | 27.14% | 53.46% | 18.49% | 15.04% |
| S3 | 59.21% | 12.44% | 11.09% | 62.51% | 37.02% | 29.79% |
| S4 | 54.59% | 20.34% | 19.63% | 56.57% | 30.7% | 22.95% |
| S5 | 61.32% | 26.49% | 27.17% | 59.37% | 33.32% | 26.57% |
| S6 | 56.83% | 23.58% | 23.20% | 57.43% | 29.26% | 23.58% |

1. A method for inducing proliferation and/or differentiation of pluripotent cells, hematopoietic stem/progenitor cells, or megakaryocytic progenitor cells into platelets, comprising using a platelet differentiation culture medium comprising N2 supplement and B27 supplement components.

2. (canceled)

3. (canceled)

4. The method according to claim 1, wherein the platelet differentiation culture medium comprises N2 supplement at a concentration of about 0.5% and B27 supplement at a concentration of about 1%.

5-142. (canceled)

143. The method according to claim 1, wherein the method comprises culturing cells with platelet differentiation culture medium IV comprising N2 supplement, B27 supplement, NEAA, glutamine, ITS, ascorbic acid, IL6, SCF and TPO.

144. The method according to claim 143, wherein said N2 supplement is at a concentration of about 0.5%, said B27 supplement is at a concentration of about 1%, said glutamine is at a concentration of about 1%, said ITS is at a concentration of about 1%, said ascorbic acid is at a concentration of 50-200 μM, said IL6 is at a concentration of 5-100 ng/mL, said SCF is at a concentration of 5-100 ng/mL, and said TPO at a concentration of 5-100 ng/mL.

145. The method according to claim 1, wherein the method comprises culturing cells with platelet differentiation culture medium III comprising IL3, IL6, SCF, TPO and FLt3L.

146. The method according to claim 145, wherein said IL3 is at a concentration of 5-100 ng/mL, said IL6 is at a concentration of 5-100 ng/mL, said SCF is at a concentration of 5-100 ng/mL, said TPO is at a concentration of 5-100 ng/mL and said FLt3L is at a concentration of 5-100 ng/mL.

147. The method according to claim 1, wherein the method comprises culturing cells with platelet differentiation culture medium II comprising BMP4, VEGF and SCF.

148. The method according to claim 147, wherein said BMP4 is at a concentration of 5-100 ng/mL, said VEGF is at a concentration of 5-100 ng/mL, and said SCF at a concentration of 5-100 ng/mL.

149. The method according to claim 1, wherein the method comprises culturing cells with platelet differentiation culture medium I comprising ROCK inhibitor.

150. The method according to claim 149, wherein the ROCK inhibitor is Y27632, wherein said Y27632 is at a concentration of about 1-50 μM.

151. The method according to claim 1, wherein the method comprises one or more of the following steps:
(1) seeding the hematopoietic stem/progenitor cells into the platelet differentiation culture medium III and culturing the cells for about 7 days to obtain megakaryocytic progenitor cells; and
(2) seeding the megakaryocytic progenitor cells into the platelet differentiation culture medium IV and culturing the cells for about 5-10 days to obtain platelets.

152. The method according to claim 1, wherein the method comprises one or more of the following steps:
(1) seeding pluripotent cells in the platelet differentiation culture medium I and culturing the cells for about 5-8 days to obtain embryoids;
(2) seeding the embryoids into the platelet differentiation culture medium II and culturing the cells for about 7 days to obtain hematopoietic stem/progenitor cells;
(3) seeding the hematopoietic stem/progenitor cells into the platelet differentiation culture medium III and culturing the cells for about 7 days to obtain megakaryocytic progenitor cells; and
(4) seeding the megakaryocytic progenitor cells into the platelet differentiation culture medium IV and culturing the cells for about 5-10 days to obtain platelets.

153. The method according to claim 1, wherein the method comprises one or more of the following steps:
(1) seeding pluripotent cells in the platelet differentiation culture medium I and culturing the cells for about 5-8 days to obtain embryoids;
(2) seeding the embryoids into the platelet differentiation culture medium II and culturing the cells for about 7 days to obtain hematopoietic stem/progenitor cells;
(3) seeding the hematopoietic stem/progenitor cells into the platelet differentiation culture medium III and culturing the cells for about 7 days to obtain megakaryocytic progenitor cells;
(4) seeding the megakaryocytic progenitor cells into the platelet differentiation culture medium IV and culturing the cells for about 5-10 days to obtain platelets; and
(5) adding a metalloproteinase inhibitor into the platelet differentiation culture medium IV for about 2 days.

154. The method according to claim 153, wherein said metalloproteinase inhibitor is GM6001.

155. The method according to claim 152, wherein said platelet differentiation culture medium I, platelet differentiation culture medium II, platelet differentiation culture medium III and platelet differentiation culture medium IV further comprises a basal culture medium, said basal culture medium comprises one or more selected from the group consisting of IMDM, MEM, Ham's F12, DMEM, RPMI1640, StemSpan™ SFEM II, APEL and mTeSR.

156. The method according to claim 152, wherein said platelet differentiation culture medium I further comprises mTeSR, said platelet differentiation culture medium II further comprises APEL, said platelet differentiation culture medium III further comprises StemSpan™ SFEM II and said platelet differentiation culture medium IV further comprises IMDM or Ham's F12.

157. A culture medium for culturing platelets, comprising platelet differentiation culture medium IV, wherein said platelet differentiation culture medium IV comprising N2 supplement, B27 supplement, NEAA, glutamine, ITS, ascorbic acid, IL6, SCF and TPO.

156. The culture medium of claim 155, wherein said culture medium further comprising platelet differentiation culture medium III, said platelet differentiation culture medium III comprising IL3, IL6, SCF, TPO and FLt3L.

157. The culture medium of claim 155, wherein said culture medium further comprising platelet differentiation culture medium II, said platelet differentiation culture medium II comprising BMP4, VEGF and SCF.

158. The culture medium of claim 155, wherein said culture medium further comprising platelet differentiation culture medium I, said platelet differentiation culture medium I comprising ROCK inhibitor.
